(19) United States
(12) Patent Application Publication
(10) Pub. No.: US 2014/0330068 A1
(43) Pub. Date: Nov. 6, 2014

(54) METHOD FOR MODULATING THE ENTERIC NERVOUS SYSTEM TO TREAT A DISORDER

(71) Applicants: Michael J. Partsch, Belmont, CA (US); John W. Sadler, Belmont, CA (US); Pankaj J. Pasricha, Belmont, CA (US)

(72) Inventors: Michael J. Partsch, Belmont, CA (US); John W. Sadler, Belmont, CA (US); Pankaj J. Pasricha, Belmont, CA (US)

(21) Appl. No.: 14/362,068

(22) PCT Filed: Dec. 3, 2012
(86) PCT No.: PCT/US2012/067576

§ 371 Date: May 30, 2014

Related U.S. Application Data
(60) Provisional application No. 61/565,989, filed on Dec. 2, 2011.
(60) Provisional application No. 61/565,987, filed on Dec. 2, 2011.

(51) Int. Cl. A61N 2/00; A61N 5/00; A61N 2/02; A61N 7/00

(52) U.S. Cl. CPC A61N 2/006; A61N 7/00; A61N 5/00; A61N 2/02; A61N 2007/0026
USPC 600/13; 601/2; 607/101; 600/9

(57) ABSTRACT

Non-invasive and/or natural orifice procedures, apparatus and methods for modulating the enteric nervous system using one or more energy modalities. A system may include a probe for insertion into a natural orifice (e.g., nasogastrically, orogastrically, nasojejunally) with or without a tube to therapeutically apply energy to modulate one or more portions of the enteric nervous system. The methods described herein may provide energy to stimulate the enteric nervous system to treat disorders such as irritable bowel syndrome, constipation, depression, anxiety and/or to enhance gastric motility, intestinal motility or other neurological responses.

### CROSS REFERENCE TO RELATED APPLICATIONS

This application claims the benefit under 35 U.S.C. 119 of U.S. Provisional Patent Application No. 61/565,989, filed Dec. 2, 2012, titled “METHOD FOR MODULATING THE ENTERIC NERVOUS SYSTEM,” and U.S. Provisional Patent Application No. 61/565,987, filed Dec. 2, 2012, titled “METHOD FOR TREATING A GASTROENTEROLOGICAL CONDITION,” each of which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The systems, devices and methods described herein are in the technical field of neuromodulation and gastroenterology. More particularly, the systems, devices and methods described herein employ directed energy to the enteric nervous system to create a desired effect, such as to relieve a medical or clinical condition. In other aspects, these systems, devices and method may have non-medical uses.

### BACKGROUND

Currently, neuromodulation and neurostimulation of the enteric nervous system is achieved by implanting microelectrodes to affect a specific neuron or group of neurons. The polarization or depolarization of this neuron or group of neurons can result in an action potential. This action potential can be the direct desired result, for instance if the action potential resulted in the contraction or relaxation of a muscle fiber. Alternatively, this action potential can be propagated to another nerve either in the peripheral nervous system or central nervous system. When transmitted to these other portions of the nervous system the action potential results in the desired neural effect. Other current approaches to modifying the behavior of the enteric nervous system involve the creation of lesions in targeted areas using ablative or heating energy.

Unfortunately, existing methods of modifying the enteric nervous system in this matter are undesirable because they are complicated, overly invasive, and may result in damage to surrounding tissues. Further, they may not be sufficiently effective, and their effect may not be long lasting. Thus, it would be desirable to provide methods and systems for stimulating the enteric nervous system that can be performed non-invasively, minimally invasively and/or through a natural body orifice, such as the mouth or nose. Described herein are systems (including apparatuses and devices) and methods for neuromodulation of the enteric nervous system.

Examples of neuromodulation or neurostimulation techniques that are used in other body regions include: Transcranial Magnetic Stimulation (TMS), deep brain stimulation (DBS), Vagus nerve stimulation (VNS), spinal cord stimulation (SCS), retinal implants, and cochlear implants. Each of these stimulation methods includes features, advantages, and disadvantages that make them difficult to adapt to the enteric nervous system.

For example, transcranial magnetic stimulation is a noninvasive method to excite the elementary unit of the nervous system; neurons in the brain. Weak electric currents may be induced in the tissue by rapidly changing magnetic fields (electromagnetic induction). This way, brain activity can be triggered with minimal discomfort, and the functionality of the circuitry and connectivity of the brain can be studied. Repetitive transcranial magnetic stimulation is known as rTMS and can produce longer lasting changes. Numerous small-scale pilot studies have shown it could be a treatment tool for various neurological conditions (e.g. migraine, stroke, Parkinson's disease, dystonia, tinnitus) and psychiatric conditions (e.g. major depression, auditory hallucinations).

Deep brain stimulation is a surgical treatment involving the implantation of microelectrodes that send electrical impulses to specific parts of the brain. A deep brain stimulation system may consist of three components: an implanted pulse generator (IPG), lead, and extension. The IPG is typically a battery-powered neurostimulator, encased in a biocompatible housing, which sends electrical pulses to the brain to modulate neural activity at the target site. The lead may be a coiled wire insulated in polyurethane with four platinum iridium electrodes and is placed in one of three areas of the brain. The lead can be connected to the IPG by the extension, an insulated wire that runs from the head, down the side of the neck, behind the ear to the IPG, which is placed subcutaneously below the clavicle or in some cases, the abdomen. The IPG can be calibrated by a neurologist, nurse or trained technician to optimize symptom suppression and control side effects.

Vagus nerve stimulation is an adjunctive treatment for certain types of intractable epilepsy and major depression. VNS uses an implanted stimulator that sends electric impulses to the left Vagus nerve in the neck via a lead wire implanted under the skin. VNS implantation devices consist of a titanium-encased generator about the size of a pocket watch with a lithium battery to fuel the generator, a lead wire system with electrodes, and an anchor tether to secure leads to the vagus nerve.

Spinal Cord Stimulation is used to exert pulsed electrical signals to the spinal cord to control chronic pain. Spinal cord stimulation, in the simplest form, consists of stimulating electrodes, implanted in the epidural space, an electrical pulse generator, implanted in the lower abdominal area or gluteal region, conducting wires connecting the electrodes to the generator, and the generator remote control. SCS has notable analgesic properties and, at the present, is used mostly in the treatment of failed back surgery syndrome, complex regional pain syndrome and refractory pain due to ischemia.

A retinal implant is a biomedical implant technology currently being developed by a number of private companies and research institutions worldwide. The implant is meant to partially restore useful vision to people who have lost their vision due to degenerative eye conditions such as retinitis pigmentosa or macular degeneration. The technology consists of an array of electrodes implanted on the back of the retina, a digital camera worn on the user's body, and a transmitter/image processor that converts the image to electrical signals and beams them to the electrode array in the eye. The technology, while still rudimentary, would allow the user to see a scoreboard type image made up of bright points of light viewed from about arm's length. There are two types of retinal implants currently showing promise in clinical trials: epiretinal implants (on the retina) and subretinal implants (behind the retina). Epiretinal implants sit on top of the retina, directly stimulating ganglia using signals sent from the external camera and power sent from an external transmitter, where subretinal implants sit under the retina, stimulating bipolar or ganglion cells from underneath. Some subretinal implants use signals and power from external circuitry, while others use only incident light as a power source and effectively replace damaged photoreceptors leaving all other structures within the eye untouched. However, due to a lack of an external power source, the image signal in this second type of subretinal implant may not be as strong as that given by an externally powered epiretinal or subretinal implant.

A cochlear implant is a small, complex electronic device that can help to provide a sense of sound to a person who is profoundly deaf or severely hard-of-hearing. The device consists of an external portion that sits behind the ear and a second portion that is surgically placed under the skin (see figure). An Cochlear Implant has the following parts: a microphone to pick up sound from the environment; a processor operates on sounds picked up by the microphone and converts it into signals suitable for an electrode array; an electrode array, which is a group of electrodes that maps impulses from the stimulator appropriate regions of the auditory nerve via the cochlea; and an implanted power source and means for recharging it.

### The Enteric Nervous System

The enteric nervous system (ENS) is the intrinsic nervous system of the gastrointestinal tract. It contains complete reflex circuits that detect the physiological condition of the gastrointestinal tract, integrate information about the state of the gastrointestinal tract, and provide outputs to control gut movement, fluid exchange between the gut and its lumen, and local blood flow. It is the only part of the peripheral nervous system that contains extensive neural circuits that are capable of local, autonomous function. The ENS has extensive, two-way, connections with the central nervous system (CNS), and works in concert with the CNS to control the digestive system in the context of local and whole body physiological demands. Because of its extent and its degree of autonomy, the ENS has been referred to as a second brain. The roles of the ENS are much more restricted than the actual brain, and so this analogy has limited utility. The enteric nervous system is embedded in the lining of the gastrointestinal system. The neurons of the ENS are collected into two types of ganglia: myenteric (Auerbach's) and submucosal (Meissner's) plexuses. Myenteric plexuses are located between the inner and outer layers of the muscularis externa, while submucosal plexuses are located in the submucosa.

The ENS is a division of the autonomic nervous system, the other divisions being the sympathetic and parasympathetic, with which it has extensive connections.

The gastrointestinal tract has an external muscle coat whose purposes are to mix the food so that it is exposed to digestive enzymes and the absorptive lining of the intestine, and to propel the contents of the digestive tube. The muscle also relaxes to accommodate increased bulk of contents, notably in the stomach. The colon also has an important reservoir function to retain the feces until defecation. The enteric reflex circuits regulate movement by controlling the activity of both excitatory and inhibitory neurons that innervate the muscle. These neurons have co-transmitters; for the excitatory neurons these include acetylcholine and tachykinins; and for the inhibitory neurons these include nitric oxide, vasoactive intestinal peptide (VIP) and ATP. There is also evidence that pituitary adenylate cyclase activating peptide (PACAP) and carbon monoxide (CO) contribute to inhibitory transmission.

The times for passage of the contents through the gastrointestinal tract vary depending on the nature of the food, including its amount and nutrient content. The peristaltic activity of the esophagus takes food from the mouth to stomach in about 10 seconds, where the food is mixed with digestive juices. Gastric emptying proceeds over periods of approximately 1-2 hours after a meal, the liquefied contents being propelled by gastric peristaltic waves as small aspirates into the jejunum during this time. The fluid from the stomach is mixed with pancreatic and biliary secretions to form the liquid content of the small intestine, known as chyme. Chyme is mixed and moves slowly along the intestine, under the control of mixing and propulsive movements orchestrated by the ENS, while digestion and absorption of nutrients occurs. The average transit time through the human small intestine is 3-4 hours. Colonic transit in healthy humans takes 1-2 days.

Intrinsic reflexes of the enteric nervous system are essential to the generation of the patterns of motility of the small and large intestines. The major muscle movements in the small intestine are: mixing activity; propulsive reflexes that travel for only small distances; the migrating myoelectric complex; peristaltic rushes; and retropulsion associated with vomiting. The enteric nervous system is programmed to produce these different outcomes. In contrast to the intestine, peristalsis in the stomach is a consequence of conducted electrical events (slow waves) that are generated in the muscle. The intensity of gastric contraction is determined by the actions of the vagus nerves, which form connections with enteric neurons in the myenteric ganglia. The proximal stomach relaxes to accommodate the arrival of food. This relaxation is also mediated through vagus nerve connections with enteric neurons. Thus, the primary integrative centers for control of gastric motility are in the brainstem, whereas those for control of the small and large intestines are in the enteric nervous system. In most mammals, the contractile tissue of the external wall of the esophagus is striated muscle, and in others, including humans, the proximal half or more is striated muscle. The striated muscle part of the esophagus is controlled, via the vagus, by an integrative circuitry in the brainstem. Thus, although the myenteric ganglia are prominent in the striated muscle part of the esophagus, they are modifiers, not essential control centers, for esophageal peristalsis.

The smooth muscle sphincters restrict and regulate the passage of the luminal contents between regions. In general, reflexes that are initiated proximal to the sphincters relax the sphincter muscle and facilitate the passage of the contents, whereas reflexes that are initiated distally restrict retrograde passage of contents into more proximal parts of the digestive tract.

The progress of the contents in an oral to anal direction is inhibited when sympathetic nerve activity increases. To achieve this, transmission from enteric excitatory reflexes to the muscle is inhibited and the sphincters are contracted. The post-ganglionic sympathetic neurons utilize noradrenaline as the primary transmitter. Under resting conditions, the sympathetic pathways exert little influence on motility. They come into action when protective reflexes are activated.

The enteric nervous system regulates the movement of water and electrolytes between the gut lumen and tissue fluid compartments. It does this by directing the activity of secretomotor neurons that innervate the mucosa in the small and large intestines and control its permeability to ions. Neurotransmitters of secretomotor neurons are vasoactive intestinal peptide (VIP) and acetylcholine. Secretion is integrated with vasodilatation, which provides some of the fluid that is secreted. Most secretomotor neurons have cell bodies in submucosal ganglia.

Fluxes of fluid, greater than the total blood volume of the body, cross the epithelial surfaces of the gastrointestinal tract each day. Control of this fluid movement via the enteric nervous system is of prime importance for the maintenance of whole-body fluid and electrolyte balance. The largest fluxes are across the epithelium of the small intestine, with significant fluid movement also occurring in the large intestine, stomach, pancreas and gall-bladder. Water moves between the lumens of digestive organs and body fluid compartments in response to transfer of osmotically active molecules. The greatest absorption of water, 8-9 liters per day, accompanies inward flux of nutrient molecules and Na+ through the activation of nutrient co-transporters, and the greatest secretion accompanies outward fluxes of Cl⁻ and HC03⁻ in the small and large intestine, gall-bladder and pancreas. In each of these organs, fluid secretion is controlled by enteric reflexes. In the small intestine and most of the colon, the reflexes circuits are intrinsic, in the enteric nervous system. They balance secretion with absorptive fluxes, and draw water from the absorbed fluid and from the circulation. The activity of the secretomotor reflexes is under a physiologically important control from inhibitory sympathetic nerve pathways that respond to changes in blood pressure and blood volume through central reflex centers.

Local blood flow to the mucosa is regulated through enteric vasodilator neurons so that the mucosal blood flow is appropriate to balance the nutritive needs of the mucosa and to accommodate the fluid exchange between the vasculature, interstitial fluid and gut lumen. There are no intrinsic vasoconstrictor neurons. Overall blood flow to the gut is regulated from the CNS, via sympathetic vasoconstrictor neurons. The sympathetic vasoconstrictor neurons act in concert with the autonomic control of other vascular beds, to distribute cardiac output in relation to the relative needs of all organs. Thus in times of need, even during digestion, the sympathetic can divert blood flow away from the gastrointestinal tract.

Gastric acid secretion is regulated both by neurons and by hormones. Neural regulation is through cholinergic neurons with cell bodies in the wall of the stomach. These receive excitatory inputs both from enteric sources and from the Vagus nerve.

Gastric secretion of HC1 and pepsinogen in the stomach, and secretion of pancreatic enzymes, is largely dependent on vago-vagal reflexes. Enteric motor neurons are the final common pathway, but the roles of intrinsic reflexes are minor. Pancreatic secretion of bicarbonate, to neutralize the duodenal contents, is controlled by secretin, a hormone released from the duodenum, in synergy with activity of cholinergic and non-cholinergic enteric neurons. Secretion into the gall-bladder and bicarbonate secretion in the distal stomach are also nerve controlled.

Nerve fibers run close to endocrine cells of the mucosa of the gastro-intestinal tract, some of which are under neural control. For example, gastrin cells in the antrum of the stomach are innervated by excitatory neurons that utilize gastrin releasing peptide as the primary neurotransmitter. Conversely, hormones released by gastrointestinal endocrine cells influence the endings of enteric neurons. In a sense, the endocrine cells act like taste cells that sample the luminal environment, and release messenger molecules into the tissue of the mucosa, where the nerve endings are found. This is a necessary communication, because the nerve endings are separated from the lumen by the mucosal epithelium. An important communication is with serotonin (5-hydroxytryptamine, 5-HT) containing endocrine cells which activate motility reflexes. Excessive release of serotonin can cause nausea and vomiting, and antagonists of the 5-HT3 receptor are anti-nauseants.

Enteric neurons are involved in a number of defense reactions of the gut. Defense reactions include diarrhea to dilute and eliminate toxins, exaggerated colonic propulsive activity that occurs when there are pathogens in the gut, and vomiting.

Fluid secretion is provoked by noxious stimuli, particularly by the intraluminal presence of certain viruses, bacteria and bacterial toxins. This secretion is due in large part to the stimulation of enteric secretomotor reflexes. The physiological purpose is undoubtedly to rid the body of pathogens and their products. However, if the pathogens overwhelm the body's ability to cope, the loss of fluid can become a serious threat to the organism.

Signals between gut regions are carried both by hormones (such as cholecystokinin, gastrin and secretin) and by nerve circuits. Entero-enteric reflexes regulate one region in relation to others. For example, when nutrients enter the small intestine, secretion of digestive enzymes from the pancreas occurs. A series of nerve circuits that carry signals from one region of intestine, to sympathetic ganglia, and back to the gut wall provide a regulatory system that is unique to the gastrointestinal tract. Neurons with cell bodies in enteric ganglia and terminals in pre-vertebral sympathetic ganglia form the afferent limbs of these reflexes. These are known as intestinofugal afferent neurons (IFANs) (Szurszewski et al. 2002).

The gastrointestinal tract is in two way communication with the CNS. Afferent neurons convey information about the state of the gastrointestinal tract. Some of this reaches consciousness, including pain and discomfort from the gut and the conscious feelings of hunger and satiety, which are integrated perceptions derived from the gastrointestinal tract and other signals (blood glucose, for example). Other afferent signals, concerning, for example, the nutrient load in the small intestine, or the acidity of the stomach, do not normally reach consciousness. In turn, the CNS provides signals to control the intestine, which are, in most cases, relayed through the ENS. For example, the sight and smell of food elicits preparatory events in the gastro-intestinal tract, including salivation and gastric acid secretion. This is termed the cephalic phase of digestion.

Swallowed food stimulates the pharynx and upper esophagus, eliciting afferent signals that are integrated in the brainstem, and subsequently provide efferent signals to enteric neurons in the stomach that cause acid secretion and increased gastric volume, in preparation for the arrival of the food. At the other end of the gut, signals from the colon and rectum are relayed to defecation centers in the spinal cord, from which a programmed set of signals is conveyed to the colon, rectum and anal sphincter to cause defecation. The defecation centers are under inhibitory control from higher CNS regions, and inhibition that can be released when it is chosen to defecate. The other central influences are through sympathetic pathways, which have been discussed under the sections on control of motility and regulation of fluid exchange and local blood flow, above.

The ENS can be related to a number of neurological events, and modulation of the ENS may modulate or treat these events. Some of these events are desirable and some are not desirable. The ENS can directly or indirectly impact neuronal activity in the brain (CNS) as well as in the gastrointestinal (GI) system. Neuromodulation or neurostimulation of the enteric system may therefore bring about a desired neurological result within the neurological circuitry of the brain or GI system by selectively modulating the activity of ENS with a neuromodulation device as described herein.

### SUMMARY OF THE DISCLOSURE

Described herein are systems and methods for neuromodulation (including neurostimulation) of one or more selected regions of the enteric nervous system. By modulating the activity of these regions, one can elicit a specific and desired response in the gastrointestinal (GI) system. In some variations, the central nervous system (CNS) may be affected. The systems and method of modulating the enteric nervous system as described herein include in particular non-invasive stimulation, minimally invasive stimulation, and/or stimulation though a natural orifice. For example, stimulation may occur externally using a probe that delivers stimulation through the subject's torso to the nerves/ganglia of the ENS. Alternatively or additionally, stimulation may be from a probe deployed through a natural body orifice into the gastrointestinal tract (e.g., stomach and/or intestines) to the ENS.

In general, a system for ENS modulation may include several components, including but not limited to: one or more probes (e.g., neuromodulation probes) to deliver energy or signals to the targeted ENS region, an energy source, and a controller, which may include one or more processors configured to control the neuromodulation by controlling the application of energy from the probe(s) to the ENS. The controller may be configured to provide a series of stimulation pulses having amplitude, pulse width, pulse rate, repetition rate, etc. that is configured to modulate the region (or all) of the ENS as desired. For example, the controller may be configured to limit the amplitude of the stimulation or the total power applied so as to modulate the ENS without over stimulation of the surrounding muscle. The controller may also receive feedback and may dynamically adjust the stimulation based on the feedback.

The system may also include a housing for all or part of the system (e.g., the controller, power source, etc.).

As described in greater detail below, any appropriate energy modality may be used for stimulation of the enteric nervous system, including magnetic (e.g., inductive magnetic stimulation), electric (including non-ablative/non-thermal RF energy), sonic (e.g., ultrasonic), optical, and mechanical. Combinations of these may also be used. As discussed, many of these energy modalities have been used for neural stimulation of other body regions. We herein describe how to specifically configure and adapt such modalities for treatment of the ENS.

Any of the methods of treatment and systems described herein may also be configured to modulate the ENS specifically, without modulation or substantial modulation of other portions of the body, including intervening regions. In particular, the systems and method described herein may be adapted for modulation of the neurons and/or ganglia of the ENS without substantially modulating other nerves such as the vagus nerve. In some variations the vagus nerve is avoided by the methods and systems described herein.

There are a number of clinical indications which might be treated with the systems (including devices or apparatuses) and methods as described herein. Thus, the methods of modulating the ENS may be configured specifically as methods of treating any of the clinical indications mentioned herein. The target ENS nerves can send many different kinds of signals to the CNS, the GI tract or other areas of the body. These signals can result in a wide variety of experiences within the CNS, the GI tract, the immune system or other parts of the body. The results of neuromodulation of the target nerves could be used to treat any combination of physiological and or psychological conditions.

As illustrated herein, one schooled in the art of neurostimulation and the functional anatomy of the target nerves could use a device as described here to cause a specific pattern of neuromodulation in the target nerves in order to treat any number of disorders. Examples of clinical indications which could be treated may include: gastrointestinal disorders, mood disorders and autoimmune disorders. This list is not, and is not intended to be, a complete list of potential applications, but rather as a sample of the potential applications for this technology.

As mentioned, in some variations the systems and methods described herein are for use though a natural orifice of the patient. For example, a method or device may include placing a tube within the stomach, the intestines or other region of the GI tract, and using this tube to neuromodulate the enteric nervous system within the stomach, the intestines or other region of the GI tract in order to bring about a desired effect. A clinician may insert a neurostimulating probe (e.g., a lead) through an existing nasogastiric (NG) tube, nasojejunal (NJ), orogastric (OG) tube or other tube into the GI tract of a subject or patient and neuromodulate the ENS from or through the tube. Any of the probes described herein may be adapted as tubes (e.g., NG, NJ, OG, etc.) tubes, or may be adapted for use with such tubes.

In general, an enteral tube is a tube that is placed through the nose and into the stomach or the small bowel to decompress the stomach, or to assist with feeding and to provide nutrition. It is a flexible tube made of rubber or plastic, and it has bidirectional potential. There are three types of enteral tubes: nasogastric, nasoduodenal, and nasojejunal, depending on where the tube ends: in the stomach, the duodenum, or the jejunum. An enteral tube is placed for the patient who is unable to take in enough food or drink through the mouth to maintain body weight. Nasogastric (NG) tubes are usually placed for a short period of time. They can be used to remove gastric secretions, prevent abdominal bloating and vomiting, and to provide a way for feedings and medication administration.

A nasoduodenal (ND) or nasojejunal (NJ) tube may be placed for those who have trouble digesting food or who aspirate food or drink into the lungs. They are also used for patients with pancreatic diseases who need to be fed into the small bowel directly. ND and NJ tubes may not be used for all medicines and may require a pump for feedings.

Methods used for enteral tube placement include bedside procedures performed blindly, and fluoroscopic and endoscopic placement. Bedside procedures have success rates can be as low as 15%, and the procedure can be difficult and lengthy depending on the patient's body size and the operator's skill. In contrast, fluoroscopic placements have success rates greater than 90%, and can be safely and accurately performed on critically ill patients; but they expose the patients to additional radiation, as well as require extra time and costs by the clinical staff.

For example, described herein are systems for non-invasively modulating the enteric nervous system to treat a disorder, the system comprising: a neuromodulation probe configured to non-invasively deliver energy to a targeted region of a subject's torso to modulate the subject's enteric nervous system; an energy source for powering the neuromodulation probe; and a controller configured to control the application of energy from the energy source to the neuromodulation probe to modulate the enteric nervous system.

The neuromodulation probe may comprise a magnetic transducer, an ultrasound transducer, an optical (light) transducer, a mechanical transducer, or the like. The neuromodulation probe may be configured to rest against the subject's torso. In some variations the system includes a harness, mount, strap, belt, or other securing structure configured to secure the neuromodulation probe against the subject's torso during a treatment period (and in some cases between treatment periods).

The controller may generally be configured to cause the neuromodulation probe to deliver a train of pulses. For example, the controller may be configured to cause the neuromodulation probe to emit energy for non-invasively modulating the enteric nervous system by applying a magnetic field stimulation that this sub-threshold.

Also described herein are methods of modulating the enteric nervous system to treat a disorder, the method comprising: placing a neuromodulation probe against at least a portion of a subject's torso; and non-invasively applying energy to a subject's enteric nervous system from the neuromodulation probe to modulate the activity of the enteric nervous system.

The step of placing may comprise placing a neuromodulation probe having one or more magnetic coils against the subject's torso. In some variations, the step of placing comprises placing a neuromodulation probe comprising one or more ultrasonic transducers against the subject's torso.

The step of non-invasively applying energy may comprise applying magnetic energy, ultrasonic energy, light energy, mechanical energy, electrical energy, etc. In some variations, non-invasively applying energy comprises applying energy that is below the threshold for triggering an action potential in muscle surrounding target nerves of the enteric nervous system.

The step of non-invasively applying energy may comprise applying energy to treat a disorder selected from the group consisting of: gastrointestinal disorders, mood disorders and autoimmune disorders. For example, the step of non-invasively applying energy may comprise applying energy to treat a disorder selected from the group consisting of: gastroparesis, constipation, or ileus.

Also described herein are systems for modulating the enteric nervous system through a natural orifice, the system comprising: a probe comprising an energy transducer configured to deliver energy to at least a portion of the enteric nervous system through a luminal wall; an energy source for powering the probe; and a controller configured to control the application of energy from the energy source to the probe to modulate the enteric nervous system.

In some variations the probe comprises one or more of: a magnetic coil transducer, an ultrasound transducer, an RF transducer (further wherein the controller is configured to control the application of energy from the RF transducer that is non-ablative), or the like.

The system may also include a tube configured to be inserted from a natural body orifice and extend through a subject's Gastro-intestinal tract, wherein the tube is configured to allow passage of the probe. For example, the system may include a tube configured to pass the probe, further wherein the tube is configured to be inserted from a natural body orifice and extend from a subject's esophagus to the stomach or intestines. The controller may be configured to cause the probe to deliver a train of pulses. The controller may be configured to cause the probe to emit energy for non-invasively modulating the enteric nervous system by applying a magnetic field stimulation that this sub-threshold.

Also described herein are methods of modulating the enteric nervous system from a natural orifice to treat a disorder, the method comprising: passing a probe through a natural orifice of a subject and into a region of the subject's gastrointestinal tract; and non-invasively applying energy to the subject's enteric nervous system from the probe within the subject's gastrointestinal tract so that the applied energy modulates the activity of the enteric nervous system.

The method may also include visualizing delivery of a tube through the nose or mouth of a subject through the esophagus to the gastrointestinal tract of the subject, wherein the tube is configured to pass the probe. In some variations, the method includes visualizing placement a distal end portion of a tube in the stomach, the intestines or other region of the gastrointestinal tract of the subject, wherein the tube is configured to pass the probe. The method may also include visualizing placement the probe in the stomach, the intestines or other region of the gastrointestinal tract of the subject. The method may also include capturing, presenting, recording or transmitting information showing delivery of the tube in the stomach, intestines or other region of the gastrointestinal tract.

The step of non-invasively applying energy may comprise delivering energy to the enteric nervous system from the probe to stimulate the enteric nervous system. Non-invasively applying energy may comprise delivering acoustic energy, electromagnetic energy, mechanical energy, biochemical energy, or optical energy. In some variations, non-invasively applying energy comprises enhancing or inhibiting action potential firing within the enteric nervous system.

### BRIEF DESCRIPTION OF THE DRAWINGS

FIG. 1 illustrates the effect of electromagnetic fields on neurons.

FIG. 2 schematically illustrates one variation of a system for modulating the ENS as described herein.

FIG. 3 is a schematic illustrating another variation of a system for modulating the ENS as described.

FIG. 4 is a schematic illustrating another variation of a system for modulating the ENS.

FIG. 5 is a schematic illustrating another variation of a system for modulating the ENS.

FIG. 6 illustrates a variation of a system for modulating the ENS on a subject.

FIG. 7 illustrates a wearable variation of a system for modulating a subject's ENS.

FIG. 8 illustrates a variation of a system for modulating the ENS on a subject.

FIG. 9 illustrates a variation of a system for modulating the ENS on a subject.

FIG. 10 illustrates the application of a variation of a system for modulating the ENS on a subject.

FIG. 11 illustrates the application of another variation of a system for modulating the ENS on a subject.

FIG. 12 illustrates the application of another variation of a system for modulating the ENS on a subject.

FIG. 13 shows a nasogastric (NG) tube, nasejejunal (NJ) tube and nasoduodenal (ND) tube inserted into a subject, these tubes may be configured as probes and/or for passing probes.

FIG. 14 shows other exemplary tubes that may be used as part of a system for modulating the ENS.

FIG. 15 schematically illustrates one variation of a system for use a natural orifice to modulate the ENS.

FIG. 16 schematically illustrates another variation of a system for use in a natural orifice to modulate the ENS.

FIG. 17 illustrates the operation of one variation of a system for modulating a subject's ENS inserted through a natural orifice.

FIGS. 18-23 show end views of the distal end of tubes that may be used as part of the systems for modulating the ENS as described herein.

### DETAILED DESCRIPTION

Described herein are methods and systems for modulation of ENS. Any of these systems may include a probe having an energy transducer for delivering energy to modulate the enteric nervous system (ENS), a power source for powering the probe, and a controller for controlling the application of energy from the probe to the ENS. In some variations the systems are configured to deliver energy to modulate the ENS from outside of the body (e.g., by application to a region of the subject's torso) and/or from an internal site, though a natural orifice.

As used here ENS may refer to all or a portion of the enteric nervous system, including individual neurons or ganglia. In general, the ENS may be a useful therapeutic target for disorders in which it is the source of dysfunction and also for disorders in which it is the effector organ. For example, in treating distal ulcerative colitis, enemas containing the neurally active membrane stabiliser lignocaine have been shown to be effective, suggesting possible involvement of neural processes in the inflammatory process. In patients with irritable bowel syndrome and diarrhea, there is substantial evidence that the primary disorder is one of cerebral regulation of gut function, mediated through autonomic nerve pathways. Yet the use of opioid analogues, which act on enteric neurons, is invaluable to diminish large bowel motor and secretory function. In patients with spinal cord injury, the ENS is intact, but constipation is common. Treatment with gut mucosal mechanical stimulation, luminal laxatives, or systemically active drugs, all of which can act on enteric nerves, results in increased peristalsis and restoration towards normal gut function. As described herein, any of these disorders may be treated by modulating the ENS through the application of energy, including the application of energy non-invasively and/or through a natural orifice.

Thus, in general, the systems described herein may be used to treat a variety of disorders or dysfunctions or to otherwise modulate the ENS. For example a system as described herein may be used to treat gastrointestinal disorders by neuromodulation and/or neurostimulation of target nerves of the enteric nervous system in subjects who experience gastrointestinal distress or constipation. This neuromodulation or neurostimulation could result in stimulating the ENS in order to facility gastric motility which approximates the activity found in a normal or healthy individual.

The ENS can also send many different kinds of signals to the CNS or other areas of the body. These signals can result in a wide variety of experiences within the CNS, the immune system or other parts of the body. The results of neuromodulation of the target nerves could be used to treat any combination of physiological and or psychological conditions.

One schooled in the art of neurostimulation and the functional anatomy of the GI tract and the target nerves could use a device as described here to cause a specific pattern of neuromodulation in the target nerves in order to treat any number of disorders. As an example, clinical indications which could be treated by these systems include: post-operative ileus (POI), opioid-induced constipation (OIC), irritable bowel syndrome (IBS), and gastroparesis. The preceding list is not intended as a complete list of potential applications, but rather as a sample of the potential applications for this technology.

As describe in greater detail below, in some variations the systems described herein may be configured for use through a natural orifice, such as from the nose or mouth through the esophagus and into the stomach and/or intestines. Thus, in some variations the systems may include a tube or tubes, including tubes (or probes configured as tubes) that are configured as nasogastric (NG) tubes, nasojujunal (NJ) tubes, etc. In some variations the tubes described herein may be configured for use with visualization, which may enhance placement.

In some variations, a neuromodulation probe comprises a housing and an energy transducer, which may be selected from a magnetic coil, an acoustic or ultrasound transducer, an optical transducer, an electrode or set of electrodes. The transducer delivers energy which results in the depolarization or polarization of neurons in the targeted region. Note, the energy may depolarize or hyperpolarize a neuron or group of neurons without triggering an action potential; thus, “stimulation” of the ENS may refer to either or both enhancing or inhibiting firing of action potentials, and/or actually triggering one or more action potentials. Energy modes that can be delivered to result in neuromodulation or neurostimulation include, without limitation: acoustic including ultrasound, electromagnetic including radio frequency, mechanical, biochemical stimulation, and optical. In another aspect, a system for modulating the ENS may deliver biological, biochemical, chemical, or other materials that result in the depolarization or polarization of the target nerves.

In another aspect, the transducer may include some activating material (biological, biochemical, chemical, etc.) and a reservoir that holds that material.

The energy source may be an on-board battery or an external power supply.

The controller may comprise hardware, software, firmware, or some combination thereof, such as an integrated circuit that controls the action of one or more neuromodulation probes. The controller could be entirely self-contained, or it could have instructions transmitted wirelessly to it by an external controller or transmitter. The controller may comprise a microprocessor and software. The controller may also comprise signal conditioning and amplification circuitry.

In general, although the nerves of the ENS are of particular interest herein, other nerves may be target nerves using the systems described herein. Any nerves, sensory or motor, that are accessible in the abdominal cavity may potential be target nerves. However, unless specified otherwise herein, the target nerves are typically part of the enteric nervous system. In some variations nerves that are not part of the ENS are avoided. In particular, the methods and systems described herein may be configured to specifically avoid direct modulation/stimulation of the vagus nerve.

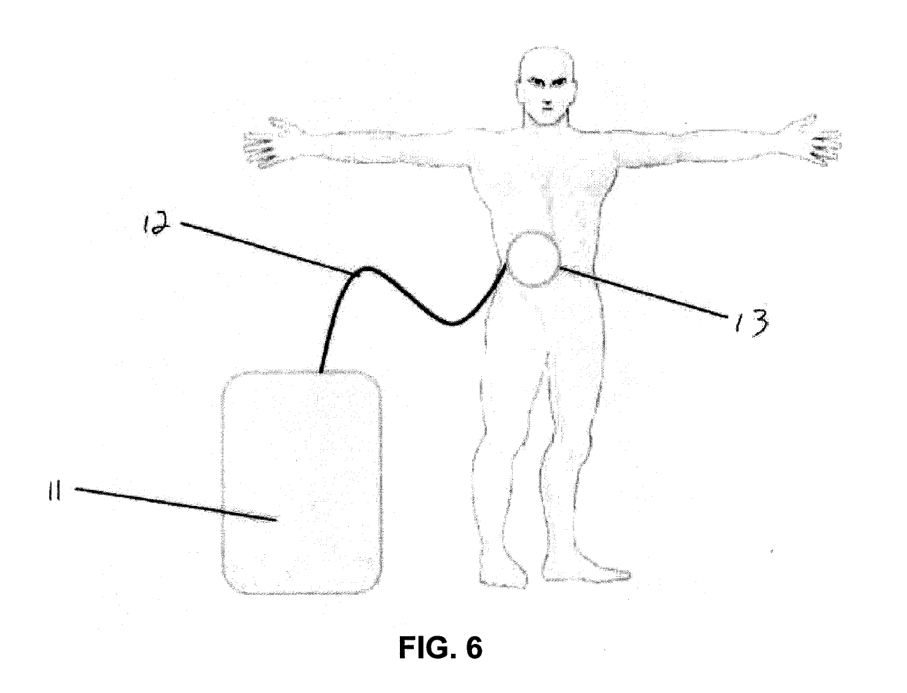

In one variation, the system contains all the elements in a single probe, which could be placed adjacent to the abdominal cavity, from any side of the body (dorsal, ventral or lateral) in order to reach the desired target nerves. This is illustrated below and shown in FIGS. 6-12. The actuator may then be in a position to send a neuromodulation signal to the respective target nerves in order to cause the desired result.

In another variation, all components of the neuromodulation system are fully housed suitable for attachment to or wearing on the abdomen.

Although the majority of the systems and methods described herein are non-invasive, in some variations the systems may be implanted or may include an implantable component. For example, in one variation, the system comprises an implanted portion and a non-implanted portion. The implanted portion would be placed within the abdominal cavity or in a subcutaneous manner adjacent or around the abdominal cavity, and could contain any or all of the components mentioned above. The external portion may control the implanted portion. This external portion could be used either by a clinician, a patient, or another individual, in order to enable the implanted portion to cause the desired neuromodulation.

In variations for use through a natural orifice, the system may include one or more tubes for positioning distally within the stomach or intestines. For example, a method of operation of such a system may include safely and accurately placing a tube in a desired location within the GI tract of a patient, recording that placement and performing neuromodulation or neurostimulation of one or more selected regions of the enteric nervous system. By modulating the activity of these regions, one can elicit a specific and desired response in either the gastrointestinal (GI) system or the central nervous system (CNS).

This system may comprise several components, including but not limited to: one or more neuromodulation probes or leads to deliver energy or signals to the targeted region, an energy source, a controller with one or more processors to control the neuromodulation process, a tube through which the leads are placed in the targeted region, a visualization system to image the process of placement, a recording system to record the images and other information from the placement process, a transmitter to convey information from the device to a receiver, and a housing.

The neuromodulation probe or lead may comprise a housing and a transducer, which may be selected from a magnetic coil, an acoustic or ultrasound transducer, an optical transducer, an electrode or set of electrodes, as mentioned above. The transducer delivers energy which may result in the depolarization or hyperpolarization of neurons in the targeted region. Energy modes that can be delivered to result in neuromodulation or neurostimulation include, without limitation: acoustic including ultrasound, electromagnetic including radio frequency, mechanical, biochemical stimulation, and optical.

In another variation, a system for modulating the ENS may deliver biological, biochemical, chemical, or other materials that result in the depolarization or polarization of the target nerves. A transducer may include some activating material (biological, biochemical, chemical, etc.) and a reservoir that holds that material.

In general, the energy source may be an on-board battery or an external power supply.

The system may include visualization (e.g., a visualization sub-system). For example, a system for modulating ENS may contain one or more fiber optic cables, an image processor and storage sub-system. In addition, the visualization sub-system may have some means to transmit information either wirelessly or through a wired connection.

A controller may comprise an integrated circuit that controls the action of one or more neuromodulation probes. The controller could be entirely self-contained, or it could have instructions transmitted wirelessly to it by an external controller or transmitter. The controller may comprise a microprocessor and software. The controller may also comprise signal conditioning and amplification circuitry.

In some variations the system contains all the elements in a single probe lead, which is housed in a tube which could be placed within the GI tract. In other variations the system contains several probes or leads within the tube which is placed in the GI tract.

In variations of system that are configured to be operated through a natural body orifice, the system may include a lead or probe which is delivered through an existing and ‘in-place’ tube within a patient (e.g., using an existing tube such as an NG tube, NJ tube, etc.

### Example of a Device for Post-Surgical Gastrointestinal Treatment

As mentioned above, enteric nerves are responsible for the motility and processing of the GI tract. Following a surgical procedure, the GI tract often does not function. This is known as post-operative ileus (POI). Significant problems can occur for patients who suffer from POI and hospitals try to facilitate intestinal motility as soon as possible post-surgery. POI can also result in significant costs for the healthcare system.

In one variation of a method to treat POI, the EMS may be modulated using a system configured to deliver energy to a portion of the EMS through a natural body orifice. For example, visualization may be used to properly place a gastric or enteral tube in the patient and the properly placed tube may be used to actively neuromodulate the ENS. This typically results in activating gastric and intestinal motility. For example, magnetic stimulation may be applied from one or more magnetic coils to modulate neurons of the ENS. In another variation, the energy applied may be ultrasound and/or electrical energy. In many cases a tube is already in place into the GI tract, and can be used to insert a probe including an energy transducer. This would allow for the prepositioned placement of a fully active neutomodulatory tube, or the insertion of a neuromodulatory lead through an existing tube.

### Example of a Device to Treat Gastroparesis

Gastroparesis, also called delayed gastric emptying, is a medical condition consisting of a paresis (partial paralysis) of the stomach, resulting in food remaining in the stomach for a longer period of time than normal. Gastroparesis may occur when the vagus nerve is damaged and the muscles of the stomach and intestines do not work normally. Chronic gastroparesis is frequently due to autonomic neuropathy. This may occur in people with type 1 diabetes or type 2 diabetes.

An ENS neuromodulation system may be used to treat gastroparesis. For example, an NG tube may be placed (or may already be positioned) into the stomach of the patient. Once in place, the NG tube could be activated to begin neuromodulation of the ENS. The result would be an increase in the rate of the gastric emptying in the subject.

Alternatively, if the patient had a venting or feeding port in place, which is common in diabetes patients, the clinician could insert an active tube in through the venting port. The ENS could be simulated by applying energy that is below the threshold of stimulation to activate muscle contraction, however it may be sufficient to at least slightly depolarize neurons of the ENS. The stimulation may be applied as pules of energy (e.g., a train of pulses) for any appropriate on-time period (e.g., msec, second, minutes, hours).

### Example of a Device to Treat Opioid Induced Constipation (OIC)

As mentioned above, the ENS regulates gastric and intestinal motility. Patients receiving opiates for pain often experience a complete or partial shutdown of their GI tract. This is known as Opioid-Induced Constipation (OIC). These patients are often bed-ridden, partially conscious or completely unconscious. OIC is a significant problem for patients and a burden on the healthcare system.

A system as described herein may be used to modulate the ENS to treat OIC. For example, in one variation a gastric or enteral tube may be positioned in the patient and visualization may be used the properly placed tube to actively neuromodulate the ENS. Modulation may result in activating gastric and intestinal motility. In many cases a tube may already be in place as part of another procedure; this would allow for the prepositioned placement of a fully active neutomodulatory tube, or the insertion of a neuromodulatory lead through an existing tube.

As mentioned, the enteric nerves are responsible for the motility and processing of the GI tract. The enteric nervous system (ENS) is central to normal gut function and is involved in most, if not all, disorders of the luminal gastrointestinal tract. The primary pathology of esophageal and gastrointestinal disorders can lie in the ENS, in adjacent structures such as enteric smooth muscle and gut epithelium, or in extrinsic nerves (local, spinal, and cerebral) controlling gut function. Even if the primary pathology lies in another part of the gut, such as in the mucosa, or outside the gastrointestinal tract, such as in the extrinsic neural pathways, the ENS still serves as the effector neural controller, leading to a disturbance in gut function and generation of symptoms.

The ENS, therefore, serves as a useful therapeutic target for disorders in which it is the source of dysfunction and also for disorders in which it is the effector organ. An example of the use of these ENS modulation systems to treat gastrointestinal disorders may include a probe including an energy transducer used in order to neuromodulate or neurostimulate the target nerves of the enteric nervous system in subjects who experience gastrointestinal distress or constipation. This neuromodulation or neurostimulation could result in stimulating the ENS in order to facility gastric motility which approximates the activity found in a normal or healthy individual.

### Example of a Mood Disorder Treatment

There is strong evidence that the enteric nervous system is linked to the mood disorders commonly associated with the central nervous system. The ENS informs our state of mind in many ways. Butterflies in the stomach, signaling in the gut as part of our physiological stress response, is but one example. Although gastrointestinal (GI) turmoil can sour one's moods, everyday emotional well-being may rely on messages from the ENS below to the CNS above. For example, electrical stimulation of the vagus nerve, a useful treatment for depression, may mimic these signals and be a method for treating depression and any of the other mood related disorders.

The systems described herein may be used to neuromodulate or neurostimulate the ENS, thus triggering a response in the vagus nerve, in order to treat depression or other mood disorders.

An alternative modulation pathway could involve using the technology to neuromodulate the ENS in order to regulate the production and distribution of a neurotransmitter, such as serotonin. Serotonin is an important neurotransmitter for the regulation of neural activity in the management and treatment of various mood disorders, as evidenced by the significant use of SSRI's (Selective Serotonin Reuptake Inhibitors) used as antidepressants in the treatment of depression, anxiety disorders, and some personality disorders. By regulating the production of such a neurotransmitter, one could use the technology to treat any one of several neurological and/or mood disorders where such a neurotransmitter is involved.

### Example of an Autoimmune Treatment

There is strong evidence that the enteric nervous system and the autoimmune system are linked. Two-way communication occurs between the enteric nervous system and the immune system of the gastrointestinal tract, that is, transmitters released by the terminals of enteric neurons in the mucosa influence immune-related cells, such as mast cells, and the cells of the mucosa release active substances, including cytokines and mast cell tryptase, that act on enteric neurons (De Giorgio et al. 2004; Lomax et al. 2006). The inter-communication that occurs in disorders such as Crohn's disease and ulcerative colitis are complex. The systems described herein may be used to apply a pattern of energy to trigger neuronal activation or suppression of the immune system by the enteric nervous system. In one example, the result could be a suppression of immune response against the host individual where that immune response was causing a disorder or negatively impacting the individual. In another instance, the result could be an activation of the immune response in order to facilitate a positive outcome for the host individual.

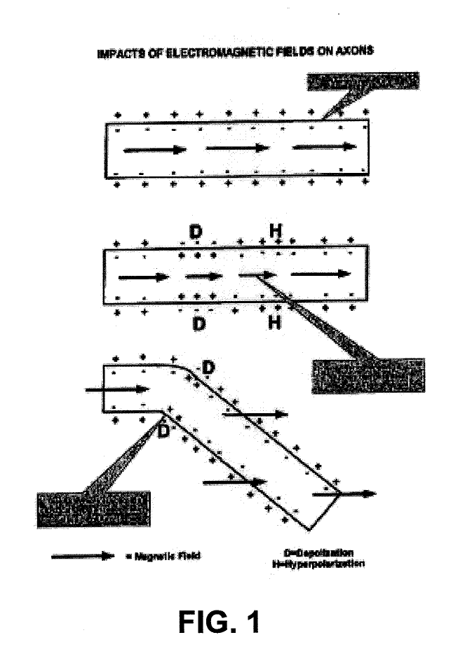

FIG. 1 illustrates a general representation of a mechanism by which the systems described herein may apply at least one energy modality (in this example, magnetic energy) to create magnetic fields which may cause depolarization of neurons. Stimulation may be most effective when the direction of the magnetic field is in the same direction of the given nerve fiber, as long as there is a magnetic-field gradient. For example, FIG. 1 shows electromagnetic effects on some axon configurations. At rest, the outside of the membrane of the nerve axon is electrically positive with respect to the interior. As shown in the top example, even if a magnetic field (shown by the heavy arrows) that is static (indicated by the arrows being of equal length) is applied, there is no depolarization of the axon and thus no nerve conduction.

However, as shown in the middle example, if there is a field gradient (indicated by the heavy arrows representing the magnetic field being of unequal length), depolarization (or at least a change in the resting potential) of the nerve membrane can occur. In such situations, hyper-polarization can occur as well. As shown in the lower example, at the location of a bend in an axon, depolarization of the membrane can occur even in the presence of a static electromagnetic field. Note that the heavy arrows are of equal length like those in the top example. The gradient of magnetic field along a straight axon and the lack of the requirement for a gradient across a bent axon work because in both cases there exists a spatial derivative of the field. Additionally, the movement of the coil in and of itself results in movement of the magnetic field and thereby may contribute to depolarization or hyperpolarization of neurons. A sensor-laden phantom, or effigy of a human target nerve system, may be used to study the effects of the neuromodulation device and to modify or otherwise optimize parameters. Neural modeling may play an important role in the functionality of the phantom, and in the planning for a specific procedure with the methods and systems described herein. Stimulation of nerve cells is not generally at the level of the individual neuron. Instead it is at the level of axon bundles or ganglia. Further, not all locations along such axon bundles are equally susceptible to electromagnetic stimulation. The points along the bundles that have the lowest threshold for excitation are where the bundles bend. Bundles may range in size from a few axons to several millimeters in size. Bundles of neurons connecting such areas may be activated using the neuromodulation device.

One aspect of repetitive stimulation and resultant discharge of the neurons may be the ability to produce long-term effects. In the brain, for example, repetitive TMS (Transcranial Magnetic Stimulation) stimulation at low frequencies can result medium-term neuronal depression while high-frequency excitation can result in medium-term potentiation. Medium term in this context means hours.

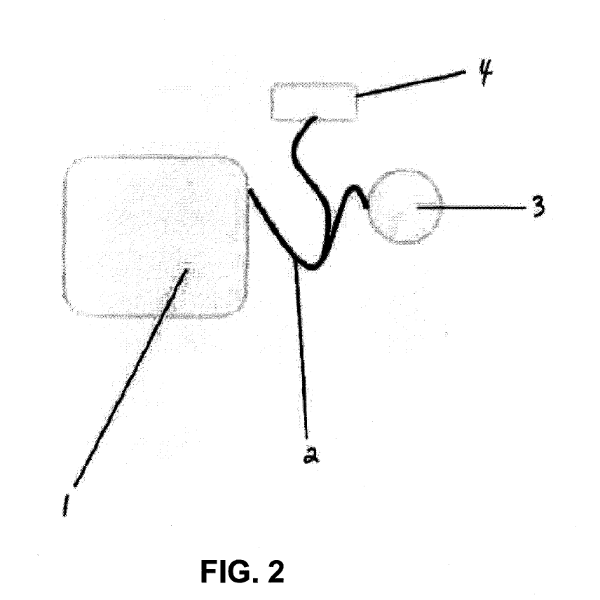

FIGS. 2-5, illustrate variations of systems that may be used to non-invasively modulate the enteric nervous system. In any of these examples the probe may include one or more transducers, as discussed above. For example, FIG. 2 shows one embodiment of a system for modulating the ENS including a probe/transducer and a controller. In FIG. 2, the energy source 1, is connected by a connector/controller wire or cable 2 to the transducer or energy probe 3, and a controller/processor 4 regulates the application of energy from the probe.

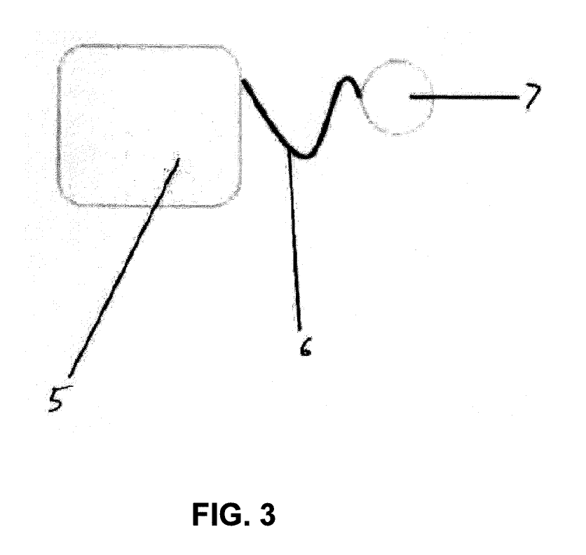

FIG. 3 shows another embodiment of a system where 5 is a combination of energy source and controller/processor in one housing, 6 is the connector/controller wire or cable, and 7 is the transducer or energy probe. The housing or probe may be configured to be applied to a particular region of the patient's anatomy (e.g., the torso).

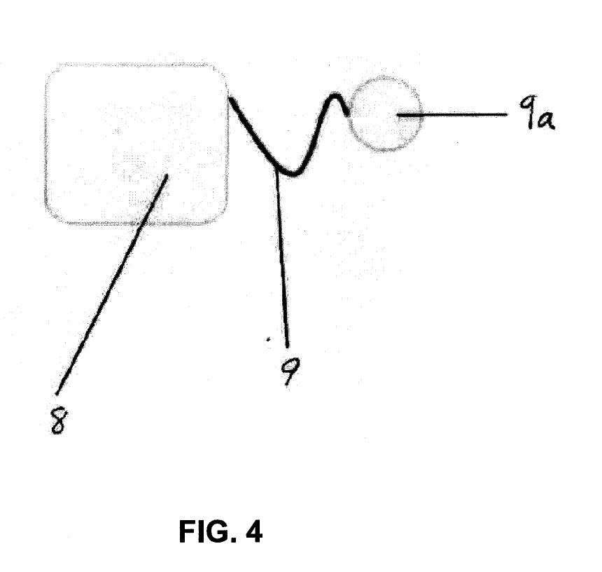

FIG. 4 shows one embodiment of a system for modulating the ENS where 8 is the energy source, 9 is the connector/controller wire or cable, and 9a is the combination controller/processor and transducer/energy probe.

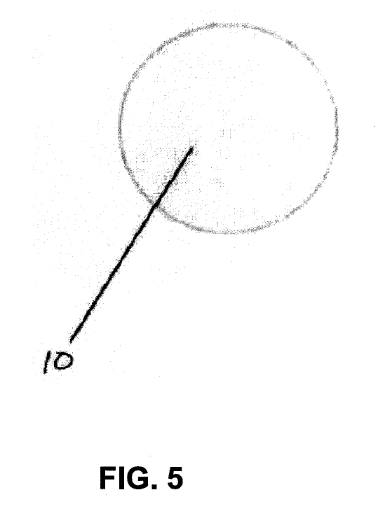

FIG. 5 shows one embodiment of a system for modulating the ENS where all components, the energy source, the controller and the transducer/probe, are housed in one unit (10).

FIGS. 6-12 illustrate methods of applying energy non-invasively from outside of the subject (e.g., on a subject's torso) to modulate the ENS. For example, FIG. 6 shows one variation a system for modulating the ENS in which where 11 is any combination of the energy source and controller, 12 is the connector/controller cable or wire, and 13 is any combination of the transducer/energy probe and possibly the controller/processor.

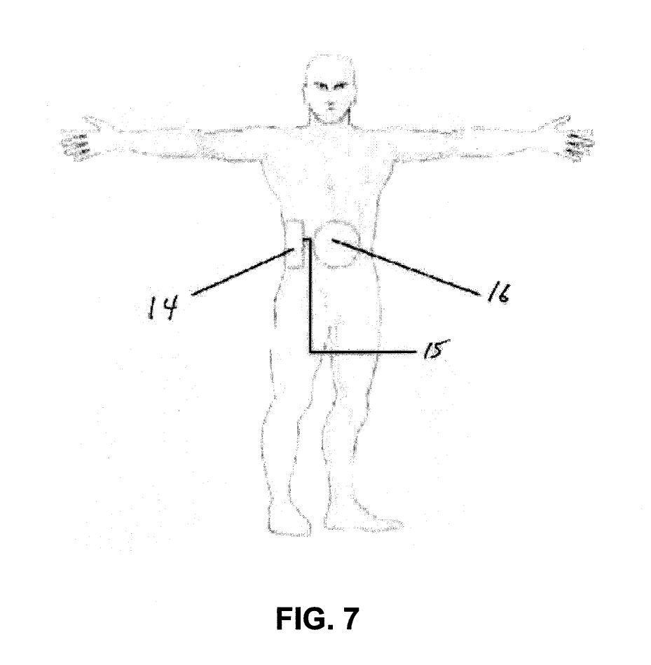

FIG. 7 shows one embodiment of a system for modulating the ENS where the system is wearable; 14 shows the housing which contains one or more of the energy source, the processor/controller and a transducer/probe. 15 is a connector/controller wire or cable. 16 is a combination of one or more of the probe, controller, and/or energy source.

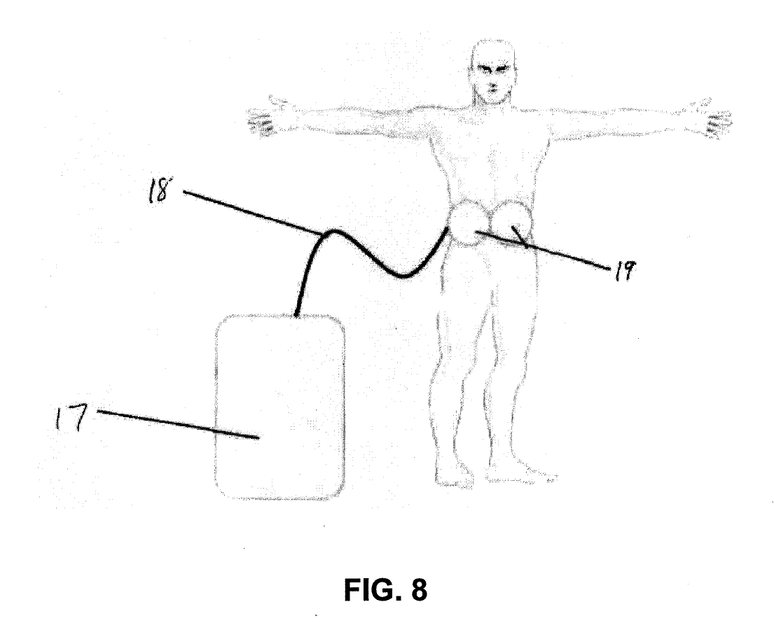

FIG. 8 shows one embodiment of a system for modulating the ENS where 17 is a housing which contains one or more of the energy source, the controller/processor and a transmitting device to send information to a potential receiver. 18 is a connector/controller wire or cable, 19 is a plurality of one or more transducers or probes.

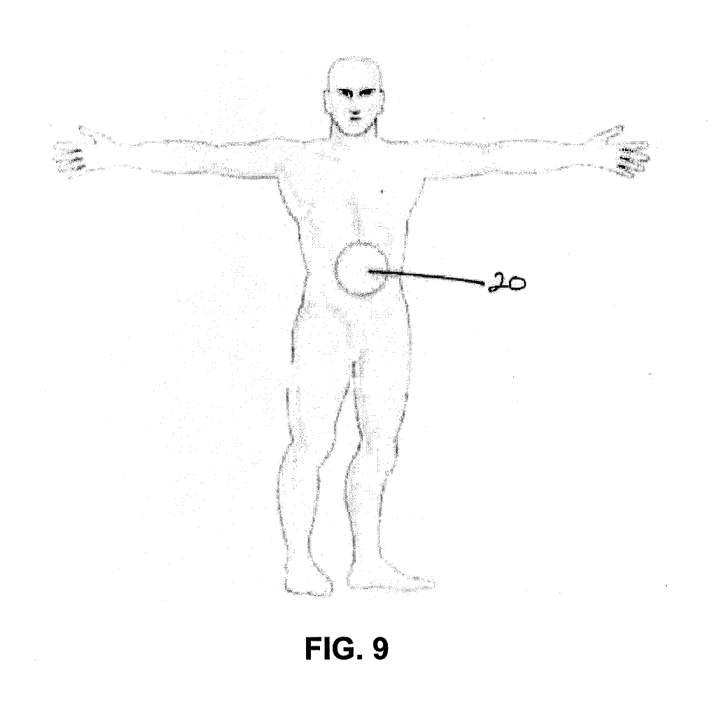

FIG. 9 shows one embodiment of a system that is a wholly contained device in one house (20) which has the energy source, the controller, the processor, the probes and any transmitter all in one housing. Note, the placement of the device may be in any location (dorsal, ventral, lateral, etc.) on or around the abdomen in order to elicit an effect.

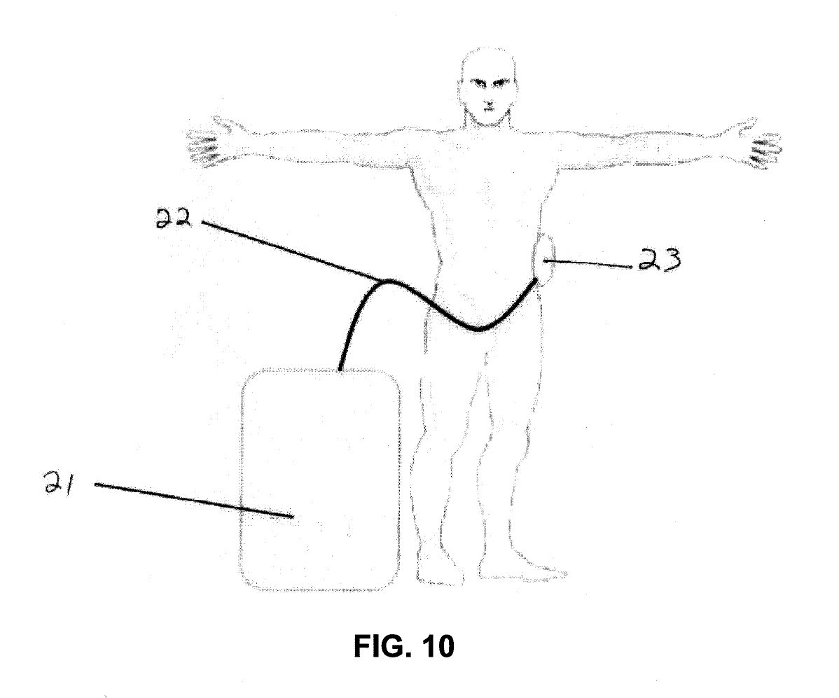

FIG. 10 illustrates a method of using a system for modulating the ENS where the probe/transducer (23) is placed along the side of the abdomen. 23 may contain any combination of probe, transmitter and processor/controller. 21 is a housing which may contain any combination of energy source, transmitter and processor/controller. 22 is a connector/controller wire or cable.

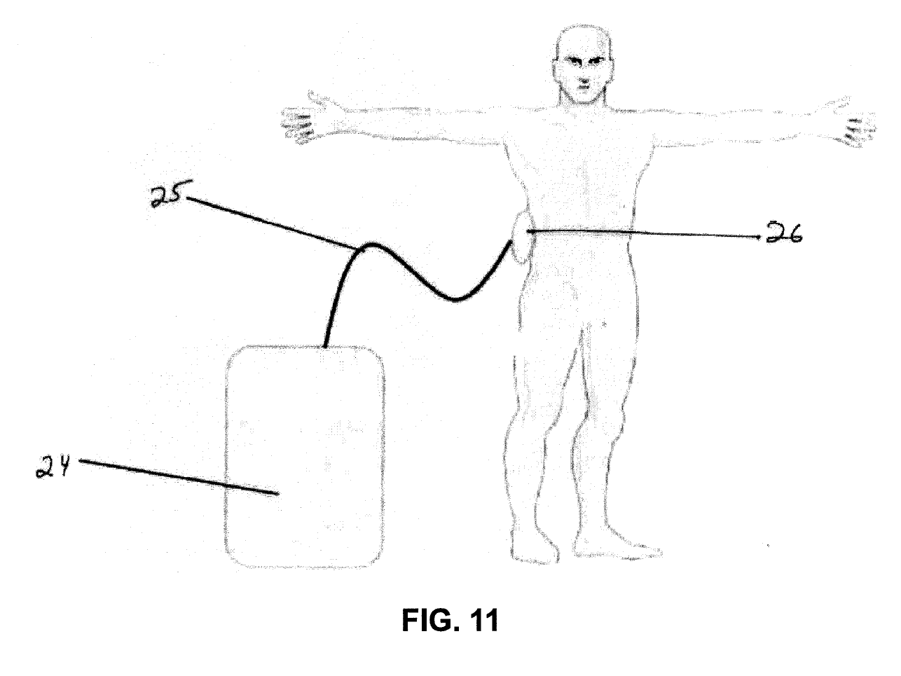

FIG. 11 illustrates a method of using a system for modulating the ENS where the probe/transducer (23) is placed along the other side of the abdomen. 26 may contain any combination of probe, transmitter and processor/controller. 24 is a housing which may contain any combination of energy source, transmitter and processor/controller. 25 is a connector/controller wire or cable.

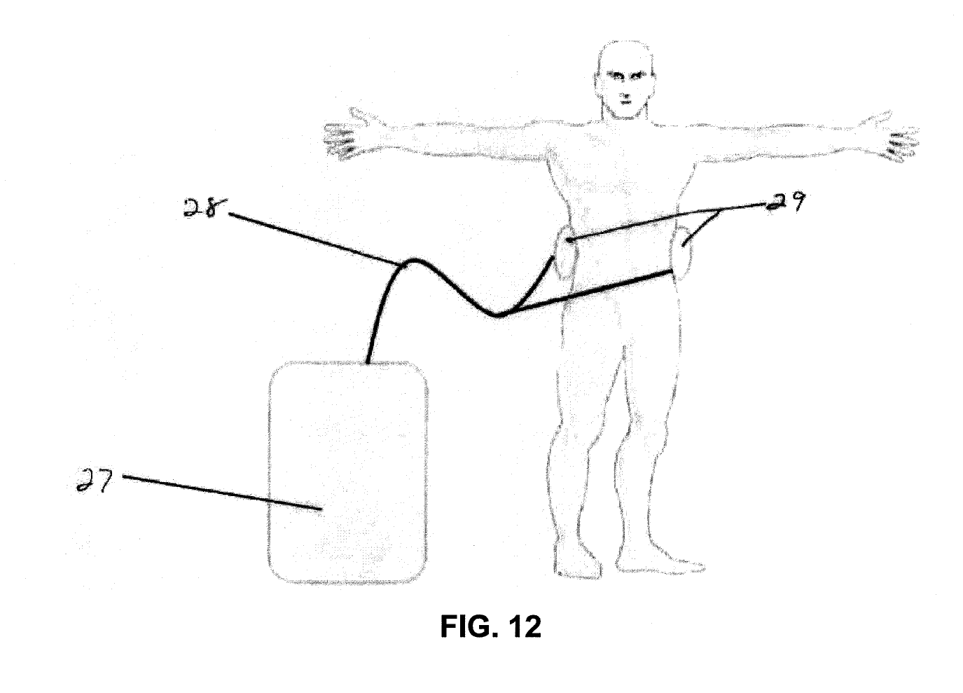

FIG. 12 illustrates a method of using a system for modulating the ENS where two probes/transducers (29) are placed along any portion of the abdomen. 29 may contain any combination of probe, transmitter and processor/controller. 27 is a housing which may contain any combination of energy source, transmitter and processor/controller. 28 is a connector/controller wire or cable. In other variations, a plurality of probes/transducers may be placed along any portion of the abdomen.

As mentioned above, any of the systems described herein may be configured so that they are inserted into a natural orifice to modulate the enteric nervous system. Such variations may be configured specifically for insertion. For example, they may be appropriately sized (e.g., having a diameter not greater than 1 inch, not greater than 0.75 inches, not greater than 0.5 inches, etc.) and flexible/conformable so that they can navigate through the esophagus and into the GI tract. As used herein the term “GI tract” typically includes the stomach and intestines.

Any of these systems may therefore be configured so that the neuromodulation probe is configured as or for use with a tube. Methods of using these systems may include steps of positioning the probe(s) within the body (e.g., within the stomach or intestines) and extending out through a natural orifice such as the nose or mouth.

Any of these systems for modulating ENS may be configured for minimally invasive or non-invasive stimulation. For example, a clinician, therapist or other care-giver may safely and accurately place a nasogastric, nasojejunal or other tube in the stomach, the intestines or other region of the GI tract of a subject or patient to deliver a probe/transducer for applying energy to modulate the ENS. The system may allow the visualization of the delivery approach, the proper placement of the tube and/or probe and a digital record of the delivery and/or placement of the tube and/or probe. Additionally, the device may allow the use of a tube to actively stimulate or neuromodulate the stomach, the intestines or other region of the GI tract. Specifically, the device may allow for the neuromodulation of the enteric nervous system (ENS). The stimulation of the ENS might be used to facilitate any number of physiological responses including the instigation, as discussed above.

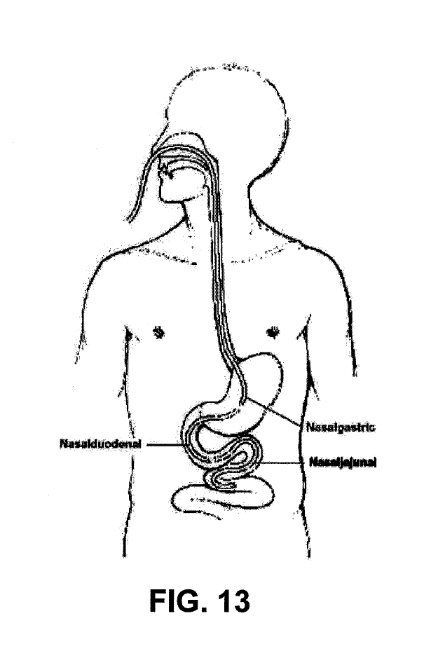

FIG. 13 shows examples of a nasogastric (NG) tube, nasejejunal (NJ) tube and nasoduodenal (ND) tube, all of which could work with the disclosed systems. As mentioned, in some variations, the systems may be configured so that the probe is itself a tube.

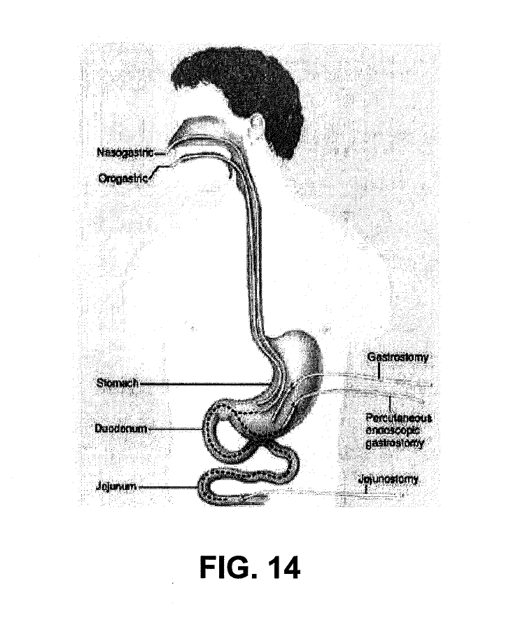

FIG. 14 shows examples of additional types of tubes which can work with the disclosed systems, including orogastric, gastrostomy, jejunostomy, and percutaneous endoscopic gastostomy (PEG).

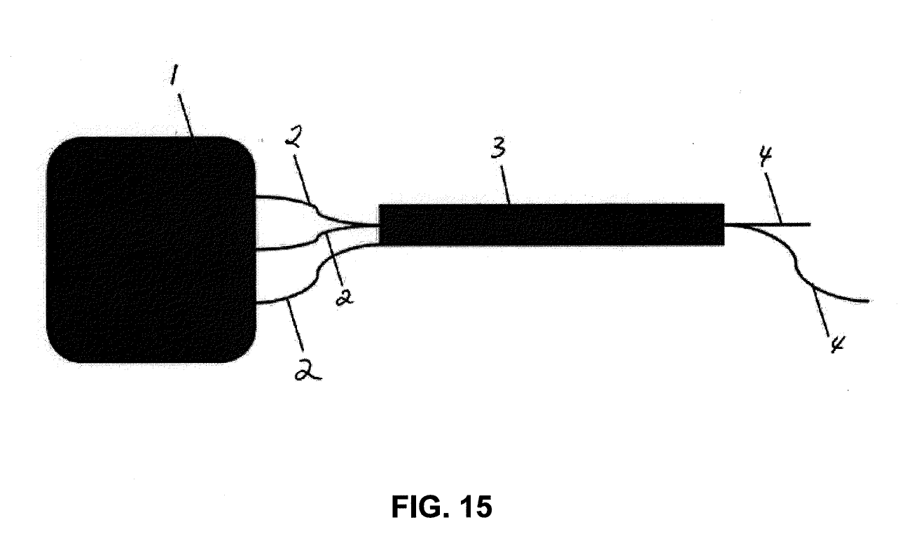

FIG. 15 shows one embodiment of a system for modulating the ENS where 1 is the combined energy source, controller power supply parts of the visual system, 2 is any number of connector/controller wires or cables which may include the control and power supply for the lead, the visual system, and other accessory functional components such as feeding tubes and aspiration tubes, 3 is the physical tube through which the lead and other components is passed, and 4 is the one or more continuations of the lead out the distal end of the tube which would be protruding into the GI tract.

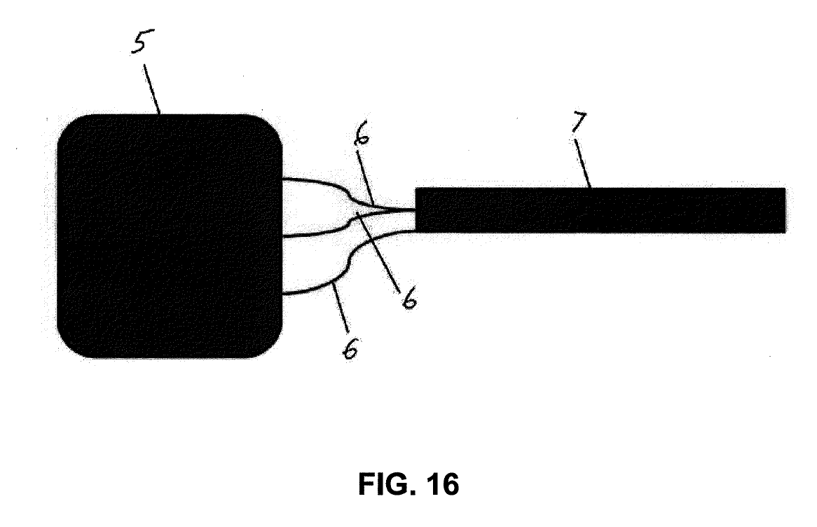

FIG. 16 shows another embodiment of a system for modulating the ENS where 5 is the combined energy source, controller power supply parts of the visual system, 6 is any number of connector/controller wires or cables which may include the control and power supply for the lead, the visual system, and other accessory functional components such as feeding tubes and aspiration tubes, and 7 is the physical tube through which the lead and other components is passed. In this embodiment, the leads and other components do not protrude through the distal end of the tube, but terminate within the tube which resides within the GI tract.

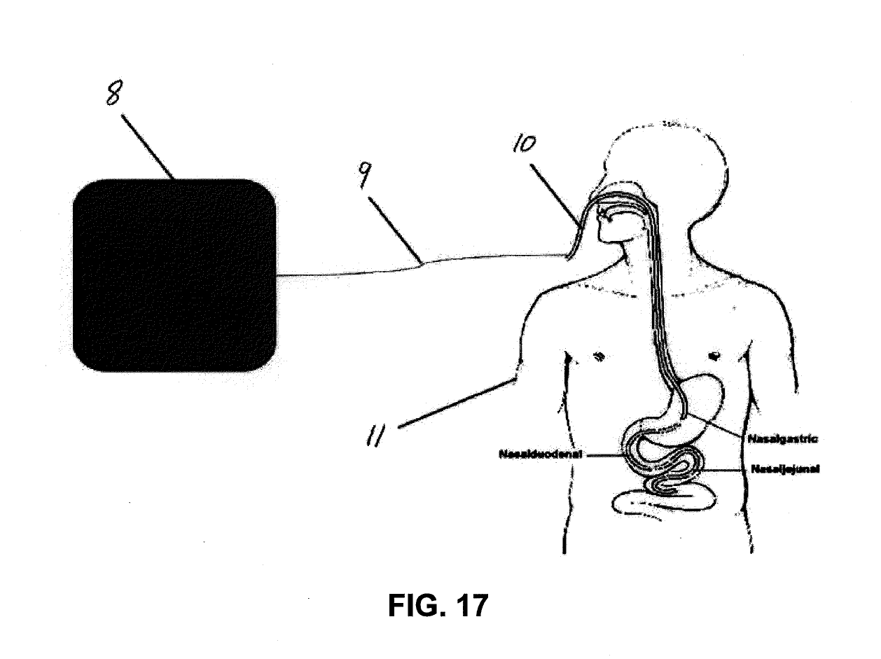

FIG. 17 illustrates the use of a system for modulating the ENS where 8 is the combined energy source, controller power supply parts of the visual system, 9 is any number of connector/controller wires or cables which may include the control and power supply for the lead or leads which can be inserted into a previously inserted NG, NJ, or ND tube (10) within a patient (11).

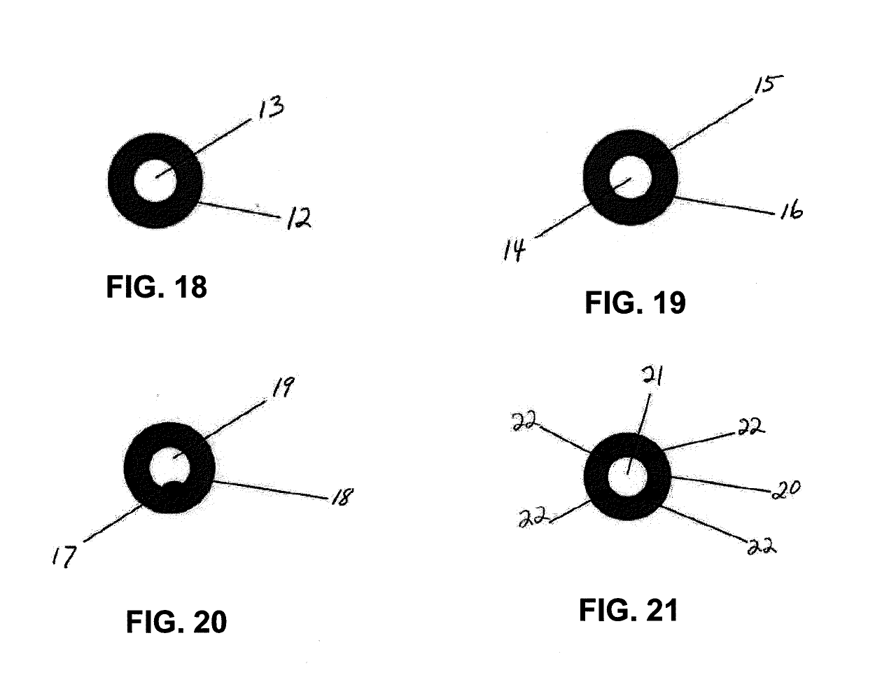

As mentioned above, any of the systems described herein may be used with a tube that may be used to introduce and/or guide a probe into proximity to the appropriate portion of the ENS to be modulated within the stomach and/or intestines. FIGS. 18-23 illustrate sections though a distal end region of various tube embodiments, including some in which the tube is also the neurostimulation probe. For example, FIG. 18 illustrates shows one embodiment of tube end on. 12 is the tube wall and 13 is the tube lumen. FIG. 19 shows one embodiment of the tube end on where 14 is the lumen, 15 is a lead or control cable placed within the wall of the tube (16).

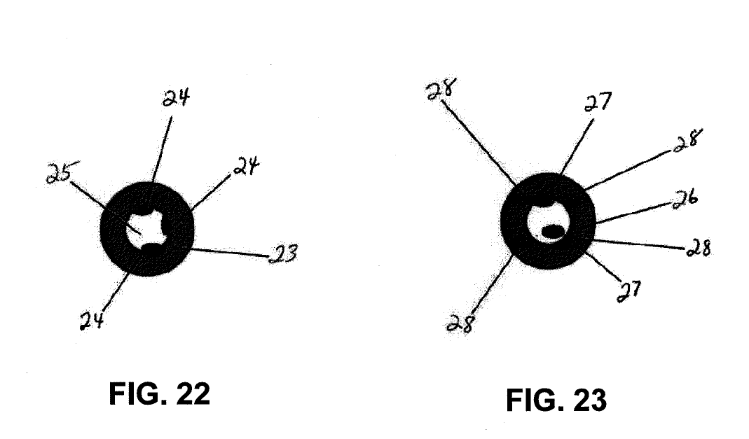

FIG. 20 shows one embodiment of the tube end on where 17 is a lead or control cable placed alongside the lumen side of the wall of the tube (18) and the lumen is 19. FIG. 21 shows one embodiment of the tube end on where 20 is the tube wall, 21 is the lumen, and 22 is any one or a plurality of leads or control cables placed within the wall of the tube (20). FIG. 22 shows one embodiment of the tube end on where 23 is the tube wall, 24 is any one or a plurality of leads or control cables placed within the lumen (25) alongside the wall of the tube (23).

FIG. 23 shows one embodiment of the tube end on where 26 is the tube wall, 27 is any one or a plurality of leads or control cables placed within the lumen alongside the wall of the tube (26), and 28 is any one or a plurality of leads or control cables placed within wall of the tube (26).

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although the foregoing systems and methods have been described in some detail by way of illustration and example for purposes of clarity of understanding, it is readily apparent to those of ordinary skill in the art in light of the teachings of this invention that certain changes and modifications may be made thereto without departing from the spirit or scope of the invention. Further, although the description above is broken into parts and includes specific examples, any of the features or elements described in any particular example or section may be incorporated into any of the other embodiments.

What is claimed is:

1. A system for non-invasively modulating the enteric nervous system to treat a disorder, the system comprising:
a neuromodulation probe configured to non-invasively deliver energy to a targeted region of a subject's torso to modulate the subject's enteric nervous system;
an energy source for powering the neuromodulation probe; and
a controller configured to control the application of energy from the energy source to the neuromodulation probe to modulate the enteric nervous system.

2. The system of claim 1, wherein the neuromodulation probe comprise a magnetic transducer.

3. The system of claim 1, wherein the neuromodulation probe comprises an ultrasound transducer.

4. The system of claim 1, wherein the neuromodulation probe is configured to rest against the subject's torso.

5. The system of claim 1, further comprising a harness configured to secure the neuromodulation probe against the subject's torso.

6. The system of claim 1, wherein the controller is configured to cause the neuromodulation probe to deliver a train of pulses.

7. The system of claim 1, wherein the controller is configured to cause the neuromodulation probe to emit energy for non-invasively modulating the enteric nervous system by applying a magnetic field stimulation that this sub-threshold.

8. A method of modulating the enteric nervous system to treat a disorder, the method comprising:
placing a neuromodulation probe against at least a portion of a subject's torso; and
non-invasively applying energy to a subject's enteric nervous system from the neuromodulation probe to modulate the activity of the enteric nervous system.

9. The method of claim 8, wherein placing comprises placing a neuromodulation probe comprising one or more magnetic coils against the subject's torso.

10. The method of claim 8, wherein placing comprises placing a neuromodulation probe comprising one or more ultrasonic transducers against the subject's torso.

11. The method of claim 8, wherein non-invasively applying energy comprises applying magnetic energy.

12. The method of claim 8, wherein non-invasively applying energy comprise applying ultrasonic energy.

13. The method of claim 8, wherein non-invasively applying energy comprises applying energy that is below the threshold for triggering an action potential in muscle surrounding target nerves of the enteric nervous system.

14. The method of claim 8, wherein non-invasively applying energy comprises applying energy to treat a disorder selected from the group consisting of: gastrointestinal disorders, mood disorders and autoimmune disorders.

15. The method of claim 8, wherein non-invasively applying energy comprises applying energy to treat a disorder selected from the group consisting of: gastroparesis, constipation, and ileus.

16. A system for modulating the enteric nervous system through a natural orifice, the system comprising:
a probe comprising an energy transducer configured to deliver energy to at least a portion of the enteric nervous system through a luminal wall;
an energy source for powering the probe; and
a controller configured to control the application of energy from the energy source to the probe to modulate the enteric nervous system.

17. The system of claim 16, wherein the probe comprise a magnetic coil transducer.

18. The system of claim 16, wherein the probe comprises an ultrasound transducer.

19. The system of claim 16, wherein the probe comprises an RF transducer, further wherein the controller is configured to control the application of energy from the RF transducer that is non-ablative.

20. The system of claim 16, further comprising a tube configured to be inserted from a natural body orifice and extend through a subject's Gastro-intestinal tract, wherein the tube is configured to allow passage of the probe.

21. The system of claim 16, further comprising a tube configured to pass the probe, further wherein the tube is configured to be inserted from a natural body orifice and extend from a subject's esophagus to the stomach or intestines.

22. The system of claim 16, wherein the controller is configured to cause the probe to deliver a train of pulses.

23. The system of claim 16, wherein the controller is configured to cause the probe to emit energy for non-invasively modulating the enteric nervous system by applying a magnetic field stimulation that this sub-threshold.

24. A method of modulating the enteric nervous system from a natural orifice to treat a disorder, the method comprising:
passing a probe through a natural orifice of a subject and into a region of the subject's gastrointestinal tract; and
non-invasively applying energy to the subject's enteric nervous system from the probe within the subject's gastrointestinal tract so that the applied energy modulates the activity of the enteric nervous system.

25. The method of claim 24, further comprising visualizing delivery of a tube through the nose or mouth of a subject through the esophagus to the gastrointestinal tract of the subject, wherein the tube is configured to pass the probe.

26. The method of claim 24, further comprising visualizing placement a distal end portion of a tube in the stomach, the intestines or other region of the gastrointestinal tract of the subject, wherein the tube is configured to pass the probe.

27. The method of claim 24, further comprising visualizing placement the probe in the stomach, the intestines or other region of the gastrointestinal tract of the subject.

28. The method of claim 25, further comprising capturing, presenting, recording or transmitting information showing delivery of the tube in the stomach, intestines or other region of the gastrointestinal tract.

29. The method of claim 24, wherein non-invasively applying energy comprises delivering energy to the enteric nervous system from the probe to stimulate the enteric nervous system.

30. The method of claim 24, wherein non-invasively applying energy comprises delivering acoustic energy, electromagnetic energy, mechanical energy, biochemical energy, or optical energy.

31. The method of claim 24 wherein non-invasively applying energy comprises enhancing or inhibiting action potential firing within the enteric nervous system.
